(19) United States
(12) Patent Application Publication
(10) Pub. No.: US 2021/0407651 A1
(43) Pub. Date: Dec. 30, 2021

(54) MEAL INFORMATION MANAGEMENT DEVICE, MEAL INFORMATION MANAGEMENT METHOD, AND NON-TRANSITORY RECORDING MEDIUM HAVING RECORDED THEREIN PROGRAM

(71) Applicant: OMRON HEALTHCARE Co, Ltd., Kyoto (JP)

(72) Inventors: Toru DENO, Kyoto (JP); Hiroshi USUI, Kyoto (JP); Miho INOUE, Kyoto (JP); Masakazu KISHIMOTO, Kyoto (JP); Tamio UEDA, Kyoto (JP); Mizuki FURUTA, Kyoto (JP)

(21) Appl. No.: 17/447,633

(22) Filed: Sep. 14, 2021

Related U.S. Application Data
(63) Continuation of application No. PCT/JP2020/008792, filed on Mar. 3, 2020.

(30) Foreign Application Priority Data
Mar. 19, 2019 (JP) 2019-051645

(51) Int. Cl. G16H 20/60; G16H 10/60; G09B 19/00; G06F 16/9535; A61B 5/021

(52) U.S. Cl. CPC G16H 20/60; G16H 10/60; A61B 5/021; G06F 16/9535; G09B 19/0092

(57) ABSTRACT

A meal information management device according to one aspect includes: a biological information acquisition unit that acquires biological information of a user; a first generation unit that generates recommended meal information related to a meal menu which satisfies a standard based on the biological information at a specific time acquired by the biological information acquisition unit; and an output unit that outputs the generated recommended meal information.

### CROSS-REFERENCE TO RELATED APPLICATIONS

This application is the U.S. national stage application filed pursuant to 35 U.S.C. 365(c) and 120 as a continuation of International Patent Application No. PCT/JP2020/008792, filed Mar. 3, 2020, which application claims priority to Japanese Patent Application No. 2019-051645, filed Mar. 19, 2019, which applications are incorporated herein by reference in their entireties.

### TECHNICAL FIELD

Aspects of the present invention relate to technology for providing meal information such as a cooking recipe to a user.

### BACKGROUND ART

Conventionally, a system for providing a cooking recipe has been proposed. For example, Patent Document 1 discloses a health maintenance support system that presents a cooking recipe for supplementing nutrients insufficient for a user. The health maintenance support system determines the deficiency of a nutrient on the basis of a comparison between the amount of each nutrient ingested by the user and a predetermined reference value.

### CITATION LIST

### Patent Literature

Patent Document 1: JP 2006-252150 A

### SUMMARY OF INVENTION

### Technical Problem

Such reference values as used in the health maintenance support system disclosed in Patent Document 1 are not set individually for the users, but are set uniformly. Thus, the effect of maintaining health may not be obtained as expected depending on the user.

The present invention has been made in view of the circumstances, and an object thereof is to provide a meal information management device, a meal information management method, and a program capable of providing users with meal information that can be expected to be effective in maintaining or improving the user's health state.

### Solution to Problem

The present invention adopts the following configurations to solve the above-described problems.

A first aspect of the present invention provides a meal information management device including: a biological information acquisition unit configured to acquire biological information of a user; a first generation unit configured to generate recommended meal information related to a meal menu that satisfies a standard based on the biological information at a specific time acquired by the biological information acquisition unit; and an output unit configured to output the generated recommended meal information.

According to the configuration, it is possible to output the meal information suitable for the user. As a result, it is possible to provide the user with meal information that can be expected to be effective in maintaining or improving the health state of the user.

In a second aspect of the present invention, the specific time is a time before a start of meal management for the user.

According to the configuration, it is possible to output meal information suitable for the user with a less burden on the user.

In a third aspect of the present invention, the specific time is included in a time range from a predetermined time period before a scheduled meal start time of the user to the scheduled meal start time.

According to the configuration, recommended meal information is generated on the basis of the biological information immediately before the meal. Therefore, it is possible to output meal information more suitable for the user. As a result, it is possible to provide the user with meal information that can be expected to be further effective in maintaining or improving the health state of the user.

In the fourth aspect of the present invention, the meal information management device further includes a standard change unit configured to change the standard on a basis of biological information acquired over a predetermined period by the biological information acquisition unit.

According to the configuration, it is possible to change the standard so that the meal information that can be expected to be further effective by maintaining or improving the health state of the user can be provided.

In a fifth aspect of the present invention, the meal information management device further includes: an attribute information acquisition unit configured to acquire attribute information related to a meal ingested by the user; a change information acquisition unit configured to acquire change information representing a change in biological information of the user before and after ingestion of the meal; an attribute information storage unit configured to store the acquired attribute information in association with the acquired change information; a selection unit configured to select attribute information associated with change information satisfying a predetermined condition from the attribute information stored in the attribute information storage unit; and a second generation unit configured to generate recommended meal information related to a meal menu that matches the selected attribute information.

According to the configuration, it is possible to output meal information related to a meal menu having the same attributes as the meal estimated to be effective in maintaining or improving the health state of the user. As a result, it is possible to provide the user with meal information that can be expected to be effective in maintaining or improving the health state of the user.

In a sixth aspect of the present invention, the second generation unit is configured to generate meal information related to a meal menu including a type and an amount of a nutritional component included in the selected attribute information as the recommended meal information.

According to the configuration, it is possible to output meal information related to a meal menu including the type and amount of nutritional components similar to the meal estimated to be effective in maintaining or improving the health state of the user. As a result, it is possible to provide the user with meal information that can be expected to be effective in maintaining or improving the health state of the user.

In a seventh aspect of the present invention, the second generation unit is configured to generate meal information related to a meal menu including a type and an amount of an ingredient included in the selected attribute information as the recommended meal information.

According to the configuration, it is possible to output meal information related to a meal menu including the type and amount of ingredients similar to the meal that is estimated to be effective in maintaining or improving the health state of the user. As a result, it is possible to provide the user with meal information that can be expected to be effective in maintaining or improving the health state of the user.

An eighth aspect of the present invention provides a meal information management method executed by a computer, the meal information management method including: acquiring biological information of a user at a specific time; generating recommended meal information related to a meal menu that satisfies a standard based on the acquired biological information at the specific time; and outputting the generated recommended meal information.

According to the configuration, the same effect as that of the meal information management device according to the first aspect can be obtained.

A ninth aspect of the present invention provides a non-transitory recording medium having recorded therein a program for causing a computer to function as each unit included in the meal information management device according to any one of the first to seventh aspects.

According to the configuration, the same effect as that of the meal information management device according to any one of the first to seventh aspects can be obtained.

### ADVANTAGEOUS EFFECTS OF INVENTION

According to the present invention, it is possible to provide a meal information management device, a meal information management method, and a non-transitory recording medium having recorded therein a program capable of providing users with meal information that can be expected to be effective in maintaining or improving the health state of the user.

### BRIEF DESCRIPTION OF DRAWINGS

FIG. 1 is a block diagram illustrating a meal information management system according to a first embodiment.

FIG. 2 is a block diagram illustrating a hardware configuration example of a server illustrated in FIG. 1.

FIG. 3 is a block diagram illustrating a software configuration example of the server illustrated in FIG. 1.

FIG. 4 is a flowchart illustrating an operation example in which the server illustrated in FIG. 1 presents recommended meal information to a user.

FIG. 5 is a flowchart illustrating an operation example in which the server illustrated in FIG. 1 changes the standard for a meal.

FIG. 6 is a block diagram illustrating a software configuration example of a server according to a second embodiment.

FIG. 7 is a flowchart illustrating an operation example in which the server illustrated in FIG. 6 registers attribute information.

FIG. 8 is a flowchart illustrating an operation example in which the server illustrated in FIG. 6 presents recommended meal information to a user.

### DESCRIPTION OF EMBODIMENTS

Hereinafter, embodiments of the present invention will be described with reference to the drawings.

### First Embodiment

### Overview

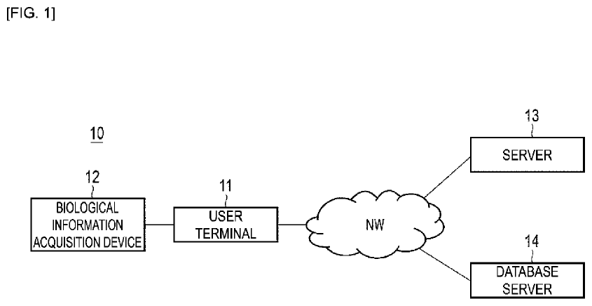

FIG. 1 schematically illustrates a meal information management system 10 according to a first embodiment. The meal information management system 10 provides meal information to a user in order to maintain or improve the health of the user. The meal information is information about a meal menu, including information representing one or more dishes. The meal information includes, for example, a cooking recipe.

As illustrated in FIG. 1, the meal information management system 10 includes a user terminal 11, a biological information acquisition device 12, a server 13, and a database server 14. The server 13 communicates with the user terminal 11 and the database server 14 via a network NW such as the Internet. The server 13 corresponds to a meal information management device of the present invention.

The user terminal 11 is a computer used by a user, such as a smartphone, a tablet, a mobile phone, a laptop computer, a desktop computer, or the like. The biological information acquisition device 12 acquires the biological information of the user by measuring. The biological information is, for example, information representing the state of a living body. Examples of biological information to be measured by the biological information acquisition device 12 include a blood pressure, electrocardiogram, a heart rate, a pulse, a body temperature, weight, a body composition, and blood glucose. The body composition indicates a percentage or an amount of tissue that constitutes the body. Examples of body composition include a body fat percentage, a muscle percentage, lean fat mass, body fat mass, muscle mass, bone mass, and a water content. The biological information acquisition device 12 may acquire a plurality of types of biological information. For example, the biological information acquisition device 12 includes an oscillometric blood pressure monitor and a weight scale, the blood pressure monitor acquires measurement values of systolic blood pressure (SBP) and diastolic blood pressure (DBP), and the weight scale acquires a measurement value of the weight. The measurement results acquired by the biological information acquisition device 12 are input to the user terminal 11.The measurement results may be input by wireless communication or may be input manually by the user.

The server 13 acquires the biological information of the user at a specific time, generates recommended meal information related to a meal menu that satisfies the standard based on the acquired biological information, and transmits the generated recommended meal information to the user terminal 11. The recommended meal information refers to the meal information recommended to the user. The user terminal 11 presents the recommended meal information received from the server 13 to the user.

The specific time is, for example, a time before the meal management for the user starts. The meal management is performed for purposes such as diet therapy or diet. The diet therapy is associated with treatment for diseases such as kidney disease, diabetes, or hypertension. The server 13 may acquire the biological information of the user from the database server 14 at the time before the start of the meal management. The database server 14 holds the test values of the biological information obtained by the tests for the user in hospitals. The tests in hospitals include a urine test, a blood test, and the like, and test results including test values for a larger number of types of biological information than those measured by the biological information acquisition device 12 can be obtained. The server 13 acquires the test values for one or more types of biological information among them. The server 13 may acquire the biological information of the user from the user terminal 11 at the time before the start of the meal management.

The standard includes information on one or more nutritional items selected in accordance with the purpose of meal management. For example, the standard includes an upper limit for the energy amount. The upper limit is set on the basis of the biological information (for example, weight) of the user. The upper limit may be set further on the basis of other indicators such as the user's age, gender, height, and the like. The standard may be set by the server 13, and may be set by a physician, a user, or the like. The server 13 generates the meal information for the meal menu whose energy amount does not exceed the upper limit as recommended meal information.

In the meal information management system 10, the server 13 generates meal information for a meal menu that satisfies the standard based on the biological information of the user as recommended meal information. In this way, it is possible to present the user with meal information suitable for the user. As a result, it is possible to provide meal information that can be expected to be effective in maintaining or improving the health state of the user.

### Configuration Example

### Server

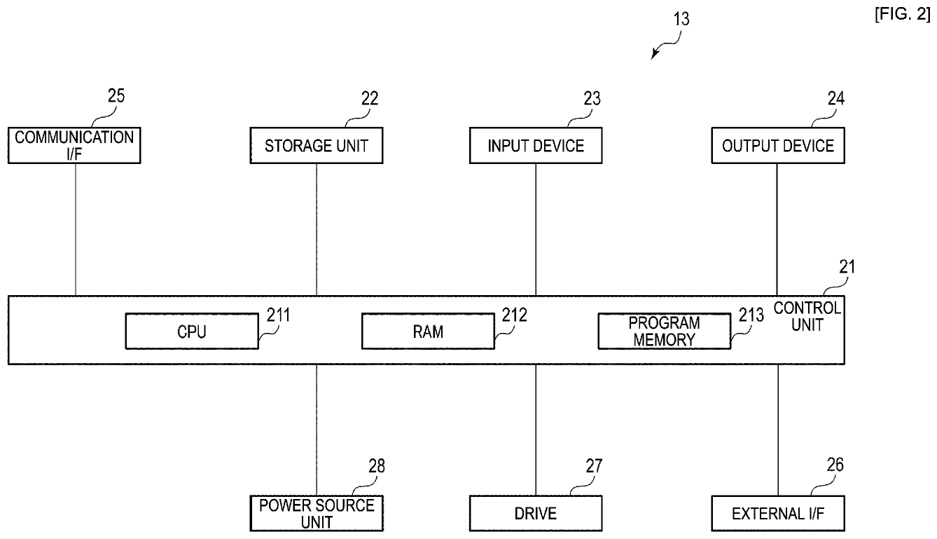

An example of the hardware configuration of the server 13 illustrated in FIG. 1 will be described with reference to FIG. 2. In the example illustrated in FIG. 2, the server 13 includes, as its hardware components, a control unit 21, a storage unit 22, an input device 23, an output device 24, a communication interface 25, an external interface 26, a drive 27, and a power source unit 28, which are electrically connected to each other.

The control unit 21 includes a Central Processing Unit (CPU) 211, a Random Access Memory (RAM) 212, and a program memory 213, and controls each component. The CPU 211 is an example of a hardware processor. The RAM 212 is a volatile memory such as a Static RAM (SRAM), and is used by the CPU 211 as a working memory. The program memory 213 stores a program such as a meal information management program and setting data necessary for executing the program. As the program memory 213, for example, a Read-Only Memory (ROM) is used. The program memory 213 may be a part of the storage unit 22.

The storage unit 22 includes a non-volatile memory such as a hard disk drive (HDD) or a solid state drive (SSD), and stores data such as meal information non-transitorily. A storage medium included in the storage unit 22 is a medium that stores data by electrical, magnetic, optical, mechanical, or chemical action so that a computer or a machine can read data such as a program.

The input device 23 is a device that receives an input. For example, the input device 23 includes a keyboard and a mouse. The output device 24 is a device that performs an output. For example, the output device 24 includes a display device and a speaker.

The communication interface 25 is an interface for communicating with an external device. For example, the communication interface 25 is used to communicate with the user terminal 11 or the database server 14 illustrated in FIG. 1. Further, the communication interface 25 may be used to download a program executed by the control unit 21 from another server (not illustrated). The communication interface 25 includes, for example, a Local Area Network (LAN) terminal, and is connected to the network NW (FIG. 1) by a LAN cable. Alternatively, the communication interface 25 may include a wireless module such as a wireless LAN module or a Bluetooth (trademark) module.

The external interface 26 is an interface for connecting an external device such as a printer. For example, the external interface 26 includes a plurality of terminals such as a Universal Serial Bus (USB) port.

The drive 27 is a device for reading data from a removable storage medium and/or writing data to the removable storage medium. The drive 27 is, for example, a semiconductor memory drive, a Compact Disk (CD) drive, a Digital Versatile Disk (DVD) drive, or the like. The drive 27 may be used to acquire a program executed by the control unit 21.

The power source unit 28 supplies electric power to the control unit 21, the storage unit 22, the input device 23, the output device 24, the communication interface 25, the external interface 26, and the drive 27. For example, the power source unit 28 includes a terminal for connecting to a commercial power supply and an AC/DC converter.

Regarding the specific hardware configuration of the server 13, components can be omitted, replaced, or added as appropriate depending on the implementation. For example, the control unit 21 may include a plurality of hardware processors.

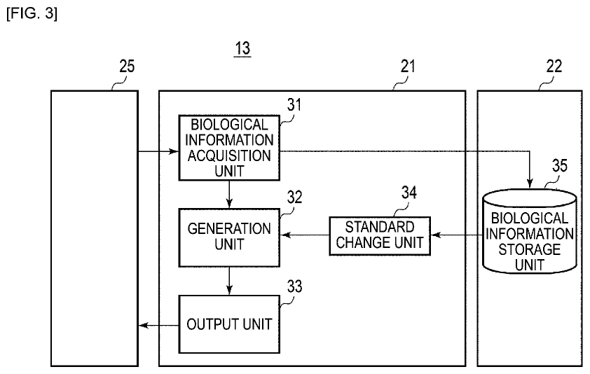

An example of the software configuration of the server 13 illustrated in FIG. 1 will be described with reference to FIG. 3. In the example illustrated in FIG. 3, the server 13 includes a biological information acquisition unit 31, a generation unit 32, an output unit 33, a standard change unit 34, and a biological information storage unit 35. The biological information acquisition unit 31, the generation unit 32, the output unit 33, and the standard change unit 34 are realized by the control unit 21 of the server 13 executing the meal information management program stored in the program memory 213. When the control unit 21 executes the program, the control unit 21 deploys the program into the RAM 212. Then, the control unit 21 interprets and executes the program deployed into the RAM 212 by the CPU 211. The biological information storage unit 35 is realized by the storage unit 22.

The biological information acquisition unit 31 acquires the biological information of the user. Specifically, the biological information acquisition unit 31 may receive data including test values related to the biological information of the user from the database server 14 via the communication interface 25. In addition, the biological information acquisition unit 31 may receive data including measurement values related to the biological information of the user from the user terminal 11 via the communication interface 25. The biological information acquisition unit 31 stores the acquired biological information in the biological information storage unit 35.

The biological information acquisition unit 31 acquires the biological information of the user at a specific time. The specific time may be, but is not limited to, a time before the start of meal management for the user. For example, when a new test value related to biological information is obtained by a test in a hospital, the new test value is acquired by the biological information acquisition unit 31 as the biological information of the user at the specific time.

The generation unit 32 generates recommended meal information on the basis of the biological information of the user at the specific time acquired by the biological information acquisition unit 31 and the standard based on the biological information. Specifically, the generation unit 32 determines a meal menu that satisfies the standard, and generates meal information related to the determined meal menu as recommended meal information. The standard may include reference values for one or more nutritional items. Examples of nutritional items include an energy amount, carbohydrates, protein, lipids, salt, sugar, and dietary fiber. Here, for the sake of simplicity, it is assumed that the standard has a reference value for one nutritional item. In one example, the standard has a lower limit for protein. In this case, the generation unit 32 generates meal information related to a meal menu in which the protein exceeds the lower limit. In another example, the standard has an upper limit for an energy amount. In this case, the generation unit 32 generates meal information related to the meal menu whose energy amount is less than the upper limit. In the example in which a database for storing the meal information associated with the energy amount is prepared, the generation unit 32 selects the meal information whose energy amount is less than the upper limit from the meal information stored in the database. When there is a plurality of pieces of meal information whose energy amount is less than the upper limit, the generation unit 32 selects at least one piece of meal information among them. In the example in which a database for storing information indicating a plurality of dishes associated with the energy amount is prepared, the generation unit 32 generates a combination of dishes (meal menu) whose energy amount is less than the upper limit, and generates meal information related to the generated combination of dishes.

The output unit 33 outputs the recommended meal information generated by the generation unit 32. Specifically, the output unit 33 transmits the recommended meal information to the user terminal 11 via the communication interface 25. The recommended meal information is presented to the user by the user terminal 11.

The standard change unit 34 changes the standard on the basis of a change in the biological information of the user. The standard change unit 34 identifies the change in the biological information of the user on the basis of the biological information stored in the biological information storage unit 35. In the example of meal management for weight loss purposes, the standard change unit 34 lowers the upper limit for the energy amount when it is identified that the weight does not tend to decrease. In the example of meal management for hypertension treatment purposes, the standard change unit 34 lowers the upper limit for salt content when it is identified that the blood pressure does not tend to decrease.

In this embodiment, an example in which all the functions of the server 13 are realized by a general-purpose processor is described. However, some or all of the functions of the server 13 may be realized by one or more dedicated processors. The biological information storage unit 35 may be provided outside the server 13, for example, in a storage unit of another server (not illustrated).

### Other

The configuration of the user terminal 11 will be described briefly. The user terminal 11 includes, as its hardware components, a control unit, a storage unit, an input device, an output device, a communication interface, an external interface, a drive, and a power source unit. The control unit includes a CPU, a RAM, a program memory, and the like. A health management application is installed on the user terminal 11. The health management application transmits the biological information acquired by the biological information acquisition device 12 to the server 13. Further, the health management application receives recommended meal information from the server 13 and presents the received recommended meal information to the user by means such as displaying the same.

The description of the specific configuration of the biological information acquisition device 12 and the database server 14 will be omitted.

### Operation Example

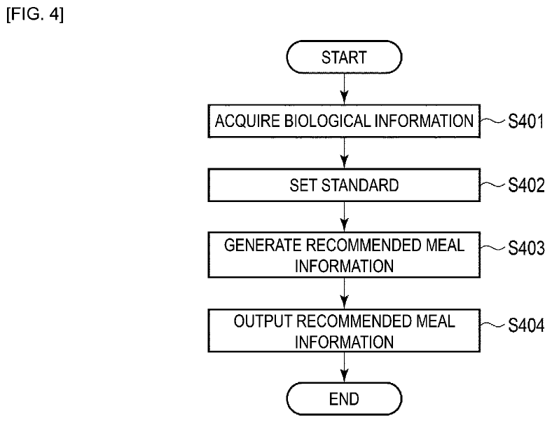

FIG. 4 illustrates an example of an operation flow when the server 13 outputs recommended meal information. In step S401 of FIG. 4, the control unit 21 of the server 13 functions as the biological information acquisition unit 31 to acquire the biological information of the user at a specific time. For example, the control unit 21 acquires the test results obtained by a test in a hospital.

In step S402, the control unit 21 sets a standard for a meal on the basis of the acquired biological information. For example, the control unit 21 determines an upper limit for the energy amount on the basis of the test value of the user's weight.

In step S403, the control unit 21 functions as the generation unit 32 to generate recommended meal information related to a meal menu that satisfies the set standard. For example, the control unit 21 generates meal information related to a meal menu whose energy amount is less than the upper limit as recommended meal information.

In step S404, the control unit 21 functions as the output unit 33 to output the generated recommended meal information to the user terminal 11 in order to present the generated recommended meal information to the user terminal 11.

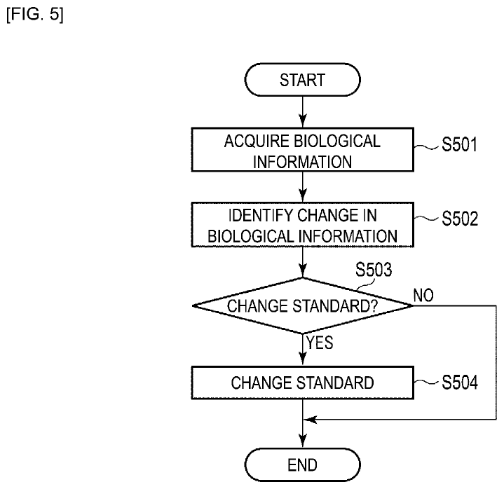

FIG. 5 illustrates an example of an operation flow when the server 13 changes the standard used when generating the recommended meal information. In step S501 of FIG. 5, the control unit 21 functions as the biological information acquisition unit 31 to acquire the biological information of the user and store the same in the biological information storage unit 35. For example, the control unit 21 acquires the measurement value related to the biological information acquired by the biological information acquisition device 12 from the user terminal 11.

In general, it is recommended that blood pressure is measured every day after wake-up and before bedtime. A blood pressure monitor as the biological information acquisition device 12 wirelessly transmits the measurement results to the user terminal 11 each time the measurement is performed, and the user terminal 11 transmits the measurement results received from the blood pressure monitor to the server 13. As a result, the blood pressure measurement values collected over a long period of time are stored in the biological information storage unit 35.

In step S502, the control unit 21 functions as the standard change unit 34 to identify a change in the biological information on the basis of the biological information stored in the biological information storage unit 35. For example, the control unit 21 identifies the change in the biological information by comparing the pieces of biological information at two time points separated by a predetermined period (for example, 30 days) or more.

In step S503, the control unit 21 functions as the standard change unit 34 to determine whether or not to change the meal standard on the basis of the identified change in the biological information. Specifically, the control unit 21 determines that the standard is not to be changed when the biological information shows an improvement tendency, and determines that the standard is to be changed when the biological information does not show an improvement tendency. For example, the control unit 21 determines that the standard is not to be changed when the weight shows a decreasing tendency, and determines that the standard is to be changed when the weight does not show a decreasing tendency. When the control unit 21 determines that the standard is not to be changed (step S503; No), the process ends. When the control unit 21 determines that the standard is to be changed (step S503; Yes), the process proceeds to step S504.

In step S504, the standard related to a meal is changed. For example, when the weight does not show a decreasing tendency, the control unit 21 lowers the upper limit for the energy amount.

The operation flows illustrated in FIGS. 4 and 5 are merely examples, and the processing procedure or processing content can be appropriately changed.

### Effects

In the meal information management system 10 described above, the server 13 generates meal information related to a meal menu that satisfies the standard based on the biological information of the user at a specific time as recommended meal information, and transmits the recommended meal information to the user terminal 11. In this way, the user terminal 11 can present the user with the meal information suitable for the user. As a result, it is possible to provide meal information that can be expected to be effective in maintaining or improving the health state of the user.

When the specific time is a time before the start of meal management for the user, it is possible to provide the user with meal information that can be expected to be effective in maintaining or improving the health state of the user with a less burden on the user.

Further, by changing the standard on the basis of the change in the biological information, it is possible to present the user with meal information more suitable for the user. As a result, it is possible to provide meal information that can be expected to be further effective in maintaining or improving the health state of the user.

### MODIFIED EXAMPLES

The specific time may be a time before ingestion of a meal. The time before ingestion of a meal is, for example, an arbitrary time within a time range from a predetermined time period (for example, one hour) before a scheduled meal start time of the user to the scheduled meal start time. The scheduled meal start time represents the scheduled time when the user starts a meal. The scheduled meal start time may be determined in advance. For example, the scheduled meal start time for lunch is determined in advance to be 12:00. The scheduled meal start time may be determined on the basis of the user's schedule. The server 13 may acquire information indicating the user's schedule from the user terminal 11. In this case, the user needs to perform the measurement with the biological information acquisition device 12 immediately before the meal, but it is possible to present the user with the meal information more suitable for the user.

### Second Embodiment

### Overview

The meal information management system according to a second embodiment provides meal information to the user in order to maintain or improve the health state of the user. The meal information management system according to the second embodiment has the same configuration as the meal information management system 10 (FIG. 1) according to the first embodiment. The meal information management system according to the second embodiment includes a server according to the second embodiment together with the user terminal 11, the biological information acquisition device 12, and the database server 14 illustrated in FIG. 1.

### Configuration Example

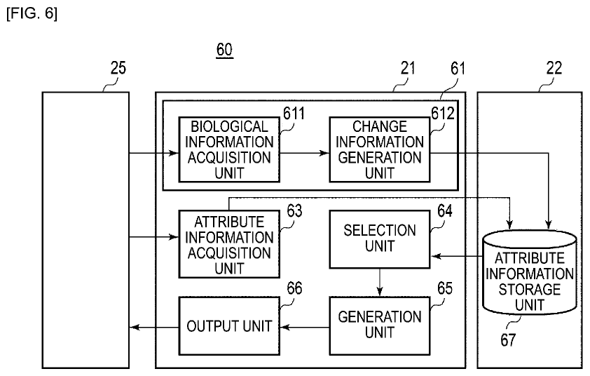

FIG. 6 schematically illustrates an example of the software configuration of a server 60 according to the second embodiment. As illustrated in FIG. 6, the server 60 includes a change information acquisition unit 61, an attribute information acquisition unit 63, a selection unit 64, a generation unit 65, an output unit 66, and an attribute information storage unit 67. The server 60 corresponds to a meal information management device of the present invention.

The server 60 may have the same hardware configuration as the server 13 according to the first embodiment. Therefore, the detailed description of the hardware configuration of the server according to the second embodiment will be omitted. As illustrated in FIG. 2, the server 60 includes a control unit 21, a storage unit 22, an input device 23, an output device 24, a communication interface 25, an external interface 26, a drive 27, and a power source unit 28. The control unit 21 includes a CPU 211, a RAM 212, and a program memory 213. The change information acquisition unit 61, the attribute information acquisition unit 63, the selection unit 64, the generation unit 65, and the output unit 66 illustrated in FIG. 6 are realized by the control unit 21 of the server 60 executing the meal information management program stored in the program memory 213. The attribute information storage unit 67 is realized by the storage unit 22.

The change information acquisition unit 61 acquires change information representing a change in the biological information of the user before and after ingestion of a meal. Specifically, the change information acquisition unit 61 includes a biological information acquisition unit 611 and a change information generation unit 612.

The biological information acquisition unit 611 acquires the biological information of the user. The biological information acquisition unit 611 receives data including measurement values related to the biological information of the user from the user terminal 11 via the communication interface 25. The biological information acquisition unit 611 acquires the biological information of the user at the time before ingestion of a meal and the biological information of the user at the time after ingestion of the meal.

The time before ingestion of the meal is, for example, an arbitrary time within a time range from a predetermined time period (for example, 30 minutes) before a meal start time to the meal start time. The meal start time refers to the time when the user starts a meal. The meal start time is, for example, input to the user terminal 11 by the user and notified to the server 13. Further, as the meal start time, the time when the meal is imaged by the camera of the user terminal 11 may be used. Further, as the meal start time, the above-described scheduled meal start time may be used.

The time after ingestion of the meal is, for example, an arbitrary time within a time range from a meal end time to a predetermined time period (for example, 30 minutes) after the meal end time. The meal end time refers to the time when the user finishes the meal. The meal end time is input by the user, for example. Further, the meal end time may be determined on the basis of the meal start time or the scheduled meal start time. For example, the meal end time is set to a predetermined time period (for example, 30 minutes) after the meal start time.

The change information generation unit 612 generates change information representing a change in the biological information of the user before and after ingestion of the meal on the basis of the biological information at a time before ingestion of the meal acquired by the biological information acquisition unit 611 and the biologic information at a time after ingestion of the meal acquired by the biological information acquisition unit 611. For example, the change information generation unit 612 calculates a difference between the biological information at the time before ingestion of the meal and the biological information at the time after ingestion of the meal, and generates change information representing the calculated difference. The change information generation unit 612 may calculate the rate of change instead of the difference.

The attribute information acquisition unit 63 acquires attribute information related to the meal ingested by the user. For example, the attribute information acquisition unit 63 receives the attribute information related to the meal ingested by the user from the user terminal 11 via the communication interface 25. The attribute information may include information indicating the type and amount of nutritional components included in the meal ingested by the user. Examples of nutritional component types include carbohydrates, protein, lipids, salt, sugar, potassium, calcium, phosphorus, and dietary fiber. The attribute information may include information indicating the type and amount of ingredients included in the meal ingested by the user. The attribute information may include information indicating the energy amount of the meal ingested by the user. As an example, the meal is imaged by the camera of the user terminal 11, and attribute information related to the meal is generated by image analysis on the user terminal 11 or the server 13.

The attribute information storage unit 67 stores the attribute information related to the meal acquired by the attribute information acquisition unit 63 in association with the change information related to the meal.

The selection unit 64 selects the attribute information associated with the change information satisfying a predetermined condition from the attribute information stored in the attribute information storage unit 67. When there is a plurality of pieces of attribute information associated with the change information satisfying the predetermined condition, the selection unit 64 selects at least one piece of attribute information among them. In one example, the predetermined condition is that the increase in blood pressure before and after a meal is less than 10 mmHg. In another example, the predetermined condition is that the increase in weight before and after a meal is less than 0.5 kg. The predetermined condition may include the conditions for a plurality of types of biological information. For example, the predetermined condition includes a condition that the increase in blood pressure before and after a meal is less than 10 mmHg, and a condition that the change in weight before and after a meal is less than 0.5 kg.

The generation unit 65 generates recommended meal information related to the meal menu that matches the attribute information selected by the selection unit 64. For example, when the attribute information selected by the selection unit 64 indicates an energy amount of 824 kcal, the generation unit 65 determines a meal menu having an energy amount of less than 824 kcal, and generates the meal information related to the determined meal menu as recommended meal information.

The output unit 66 outputs the recommended meal information generated by the generation unit 65. Specifically, the output unit 66 transmits the recommended meal information to the user terminal 11 via the communication interface 25. After that, the recommended meal information generated by the generation unit 65 is presented to the user by the user terminal 11.

Operation Example

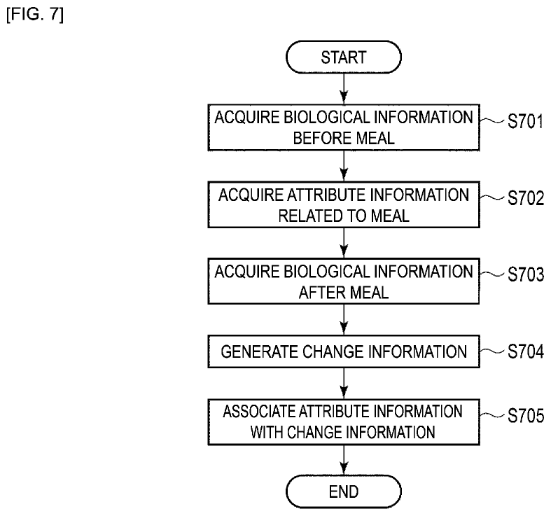

FIG. 7 illustrates an example of an operation flow when the server 60 registers the attribute information. In step S701 of FIG. 7, the control unit 21 of the server 60 functions as the biological information acquisition unit 611 to acquire the biological information of the user at the time before ingestion of the meal from the user terminal 11.

In step S702, the control unit 21 functions as the attribute information acquisition unit 63 to acquire the attribute information related to the meal from the user terminal 11 and save the acquired attribute information in the attribute information storage unit 67.

In step S703, the control unit 21 functions as the biological information acquisition unit 611 to acquire the biological information of the user at the time after ingestion of the meal from the user terminal 11.

In step S704, the control unit 21 functions as the change information generation unit 612 to generate change information representing a change in biological information before and after ingestion of a meal. For example, the control unit 21 calculates the difference between the biological information acquired in step S701 and the biological information acquired in step S703, and generates change information indicating the calculated difference.

In step S705, the control unit 21 associates the attribute information acquired in step S702 with the change information generated in step S704.

In this way, the server 60 registers the attribute information. By repeating the operation illustrated in FIG. 7 for each meal, the attribute information is accumulated in the attribute information storage unit 67.

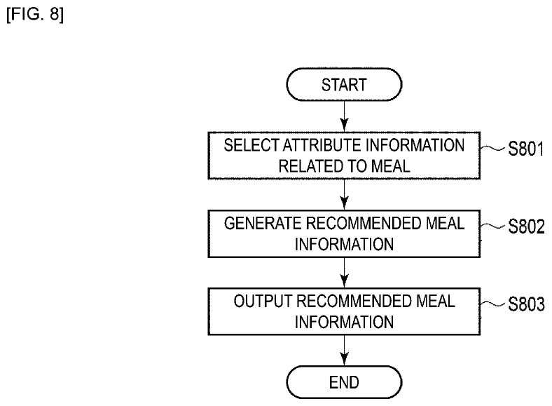

FIG. 8 illustrates an example of an operation flow when the server 60 outputs recommended meal information. The operation flow illustrated in FIG. 8 is executed, for example, 30 minutes before the scheduled meal start time.

In step S801 of FIG. 8, the control unit 21 functions as the selection unit 64 to select the attribute information associated with the change information satisfying a predetermined condition from the attribute information stored in the attribute information storage unit 67. When there is a plurality of pieces of attribute information associated with the change information satisfying the predetermined condition, the control unit 21 randomly selects one of these pieces of attribute information, for example.

In step S802, the control unit 21 functions as the generation unit 65 to generate recommended meal information related to the meal menu that matches the selected attribute information. When there is a plurality of meal menus that matches the selected attribute information, the control unit 21 randomly selects at least one of these meal menus, and generates the meal information related to the selected meal menu as the recommended meal information.

In step S803, the control unit 21 functions as the output unit 66 to output the generated recommended meal information to the user terminal 11 in order to present the generated recommended meal information to the user terminal 11. For example, when the control unit 21 generates a plurality of pieces of recommended meal information, the user terminal 11 may display a list of meal menus corresponding to these pieces of recommended meal information. Then, when any one of the meal menus is clicked, the user terminal 11 displays a cooking recipe corresponding to the clicked meal menu.

The operation flows illustrated in FIGS. 7 and 8 are merely examples, and the processing procedure or processing content can be appropriately changed.

### Effects

In the above-described meal information management system, the control unit 21 of the server 60 stores the attribute information related to the meal ingested by the user in the storage unit 22 in association with the change information representing the change in the biological information of the user before and after ingestion of the meal. The control unit 21 selects the attribute information associated with the change information satisfying the predetermined condition from the attribute information stored in the storage unit 22, and generates recommended meal information related to a meal menu that matches the selected attribute information. In this way, it is possible to output meal information related to a meal menu having the same attribute as the meal that is estimated to be effective in maintaining or improving the health state of the user. As a result, it is possible to provide the user with meal information that can be expected to be effective in maintaining or improving the health state of the user.

The control unit 21 generates meal information related to the meal menu including the type and amount of nutritional components included in the selected attribute information as recommended meal information. In this way, it is possible to output meal information related to a meal menu including the type and amount of nutritional components similar to those of a meal that is estimated to be effective in maintaining or improving the health state of the user.

The control unit 21 generates meal information related to the meal menu including the type and amount of the ingredients included in the selected attribute information as recommended meal information. In this way, it is possible to output meal information related to the meal menu including the type and amount of ingredients similar to those of the meal that is estimated to be effective in maintaining or improving the health state of the user.

### MODIFIED EXAMPLES

The attribute information storage unit 67 may store the attribute information related to the meal in association with the change information and the meal content information representing the content of the meal. In this case, the control unit 21 may generate recommended meal information related to a meal menu that matches the selected attribute information and is different from the meal content indicated by the meal content information associated with the selected attribute information. The meal content information associated with the selected attribute information may be acquired within a certain period (for example, 30 days). In this case, the user is presented with meal information related to a meal menu different from the meal content ingested by the user within a certain period. In this way, the presentation of the same recommended meal information to the user is reduced. As a result, the recommended meal information is more likely to be adopted by the user, which leads to maintenance or improvement of the health state of the user.

### Other Embodiment

Note that the invention is not limited to the above-described embodiments.

The first embodiment and the second embodiment may be combined. For example, the meal information management device operates according to the meal information management technique according to the first embodiment immediately after the start of meal management, and operates according to the meal information management technique according to the second embodiment when the number of pieces of attribute information stored in the attribute information storage unit 67 exceeds a predetermined number.

The meal information management device may adjust the cooking information (cooking recipe) presented to the user on the basis of information such as the user's age, gender, or amount of exercise. The amount of exercise may be the average amount of exercise, the (actually measured) amount of exercise on the day, or the amount of exercise on the day predicted on the basis of the schedule information.

The meal information management device may present a shopping list or an eating-out menu instead of the cooking recipe. The shopping list or eating-out menu includes, for example, information representing a combination of ready-made dishes with an appropriate nutritional composition or a combination of dishes provided for in-store or home-cooked meals. The shopping list or eating-out menu may further include information representing a store (a point-of-sale, a restaurant, and the like) that sells those dishes. In this case, the menu information including the nutrient information provided in advance by the store may be saved in the meal information management device (the server 13), or the meal information management device may acquire the menu information including the nutrient information each time from an external server (a server of the store) that provides the same in an accessible manner.

In the above-described embodiments, the meal information management device is implemented by one computer (server). In another embodiment, the meal information management device may be implemented by a plurality of computers. In still another embodiment, some or all of the functions of the meal information management device may be implemented by the user terminal 11. Specifically, the user terminal 11 may include at least one of the biological information acquisition unit 31, the generation unit 32, the output unit 33, the standard change unit 34, and the biological information storage unit 35. Further, the user terminal 11 may include at least one of the change information acquisition unit 61, the attribute information acquisition unit 63, the selection unit 64, the generation unit 65, the output unit 66, and the attribute information storage unit 67.

Note that the present invention is not limited to the above-described embodiments, and various modifications can be made in an implementation stage without departing from the gist. Furthermore, each of the embodiments may be implemented in combination as appropriate to the extent possible, and in this case, combined effects can be obtained. Further, the above-described embodiments include inventions at various stages, and various inventions can be extracted by an appropriate combination of the plurality of disclosed components.

The part or whole of the above-described embodiments can be described as, but not limited to, the following supplementary note.

### Supplementary Note 1

A meal information management device (13) including: a biological information acquisition unit (31) configure to acquire biological information of a user; a first generation unit (32) configure to generate recommended meal information related to a meal menu that satisfies a standard based on the biological information at a specific time acquired by the biological information acquisition unit; and an output unit (33) configured to output the generated recommended meal information.

### REFERENCE NUMERALS LIST

10 Meal information management system
11 User terminal
12 Biological information acquisition device
13 Server
14 Database server
21 Control unit
211 CPU
212 RAM
213 Program memory
22 Storage unit
23 Input device
24 Output device
25 Communication interface
26 External interface
27 Drive
28 Power source unit
31 Biological information acquisition unit
32 Generation unit
33 Output unit
34 Standard change unit
35 Biological information storage unit
60 Server
61 Change information acquisition unit
611 Biological information acquisition unit
612 Change information generation unit
63 Attribute information acquisition unit
64 Selection unit
65 Generation unit
66 Output unit
67 Attribute information storage unit

1. A meal information management device comprising:
one or more processors configured to:
acquire biological information of a user;
acquire attribute information related to a meal ingested by the user;
acquire change information representing a change in biological information of the user before and after ingestion of the meal;
store the acquired attribute information in association with the acquired change information in an attribute information storage;
select attribute information associated with change information satisfying a predetermined condition from the attribute information stored in the attribute information storage;
generate recommended meal information related to a meal menu that matches the selected attribute information; and
output the generated recommended meal information.

2. The meal information management device according to claim 1
a second generation unit wherein the one or more processors are further configured to generate recommended meal information related to a meal menu that satisfies a standard based on the biological information at a specific time that is acquired, and
wherein the specific time is a time before a start of meal management for the user.

3. The meal information management device according to claim 1,
wherein the one or more processors are further configured to generate recommended meal information related to a meal menu that satisfies a standard based on the biological information at a specific time that is acquired, and
wherein the specific time is included in a time range from a predetermined time period before a scheduled meal start time of the user to the scheduled meal start time.

4. The meal information management device according to claim 2, wherein the one or more processors are further configured to change the standard on a basis of biological information that is acquired over a predetermined period.

5. The meal information management device according to claim 1, wherein the one or more processors are further configured to generate meal information related to a meal menu including a type and an amount of a nutritional component included in the selected attribute information as the recommended meal information.

6. The meal information management device according to claim 1, wherein the one or more processors are further configured to generate meal information related to a meal menu including a type and an amount of an ingredient included in the selected attribute information as the recommended meal information.

7. A meal information management method executed by a computer, the meal information management method comprising:
acquiring attribute information related to a meal ingested by a user;
acquiring change information representing a change in biological information of the user before and after ingestion of the meal;
storing the acquired attribute information in an attribute information storage unit in association with the acquired change information;
selecting attribute information associated with change information satisfying a predetermined condition from the stored attribute information in the attribute information storage;
generating recommended meal information related to a meal menu that matches the selected attribute information; and
outputting the generated recommended meal information.

8. A non-transitory recording medium having recorded therein a program for causing a computer to execute functions in the meal information management device according to claim 1.
